(19) United States
(12) Patent Application Publication
(10) Pub. No.: US 2003/0129770 A1
(43) Pub. Date: Jul. 10, 2003

(54) Method to improve sensitivity of molecular binding assays using phase-sensitive luminescence detection

(76) Inventor: Salvador M. Fernandez, Hartford, CT (US)

Correspondence Address: ALIX YALE & RISTAS LLP, 750 MAIN STREET, SUITE 1400, HARTFORD, CT, 06103, US

(21) Appl. No.: 10/256,321

(22) Filed: Sep. 27, 2002

Related U.S. Application Data
(60) Provisional application No. 60/325,931, filed on Sep. 28, 2001.
(60) Provisional application No. 60/325,909, filed on Sep. 28, 2001.

(51) Int. Cl. G06F 19/00; G01N 33/48; G01N 33/50; G01N 33/543

(52) U.S. Cl. 436/518; 702/19

(57) ABSTRACT

An apparatus and method, which uses luminescence phase-sensitive detection, for improving the detection sensitivity of luminescence molecular recognition assays in which the analytical luminescence signal contains two components, each arising from a different state of the luminophore and each having a characteristic luminescence lifetime that is different from that of the other.

### CROSS-REFERENCE TO RELATED APPLICATIONS

This application claims the benefit under 35 U.S.C. §119(e) of United States Provisional Patent Application Serial No. 60/325,931 and United States Provisional Patent Application Serial No. 60/325,909, both filed Sep. 28, 2001.

### BACKGROUND OF THE INVENTION

The present invention relates to fluorescence assays including quenching and resonance energy transfer (FRET) assays. The invention also relates to the field of polymerase chain reaction (PCR), nucleic acid hybridization, ligand binding assays, protein-protein interaction assays, gene reporter assays, and functional cell assays.

Luminescence is used here as a general term to include all processes where electromagnetic energy in the ultraviolet, visible and infrared spectral ranges is emitted subsequent to an excitation process caused by absorption of electromagnetic radiation. Luminescence, therefore includes the processes of fluorescence and phosphorescence. Luminescent materials, examples of which include organic dyes, inorganic compounds, fluorescent proteins, semiconductor nanocrystals and luminescent polymers, are widely used as labels in a variety of biological assays because of their high detection sensitivity. We will refer to these luminescent compounds as luminophores, and more specifically as fluorophores and phosphors.

The most straightforward type of luminescent assay employs a luminophore as a simple tag or tracer. This tag may be attached covalently or non-covalently to a biomolecule or an analyte whose binding to a molecular recognition partner is to be measured. In one type of application the luminescence characteristics of the luminophore do not change upon the molecular recognition event (e.g., binding) to be detected. Since in a typical binding assay only a fraction of the labeled material is bound at the end of the reaction, measuring binding by this approach requires separation of the bound from the unbound material. Separation steps are undesirable because they add labor to the assay, may be difficult to automate and reduce throughput, which is a major concern in high-throughput screening applications.

A number of sophisticated luminescent methods and reagents have been developed to enable homogeneous luminescent assays, that is, assays that obviate the need to separate the bound from the free luminophore. These methods and luminophores rely on the occurrence of a detectable change in some measurable luminescence characteristics of the luminophore due to the molecular interaction being monitored.

One approach which forms the basis for various types of homogeneous fluorescence assays is based on the phenomenon of fluorescence resonance energy transfer (FRET). In FRET, energy is transferred from a donor fluorophore to an acceptor molecule by dipole-dipole interaction. The efficiency of energy transfer depends on the spectral overlap between the emission of the donor and the absorbance of the acceptor, on the inverse sixth power of the distance between the donor and acceptor molecule, and on their relative orientation [Forster, T. Delocalized excitation and excitation transfer. In Modern Quantum Chemistry, Istanbul Lectures, part III. Edited by Sinanoglu O. Academic Press. 1965: 93-137]. When an excited donor fluorophore transfers energy to an acceptor molecule through FRET, its fluorescence emission intensity decreases. If the acceptor molecule is fluorescent, FRET also results in an increase in acceptor fluorescence emission.

Because of the 1/R⁶ dependence on intermolecular distance, FRET occurs only when the donor and acceptor molecules are very close together. For most biologically useful fluorophores, FRET typically occurs for donor-acceptor distances in the range of 1 to 10 nm. Thus, FRET is often used to monitor the state of association of molecules. FRET assays can be designed such that an event of interest results in dissociation of the donor-acceptor pair or in association of the donor-acceptor pair. In the first case, molecular dissociation is manifested by an increase in the fluorescence emission intensity of the donor, and in the second case association is manifested as a decrease in fluorescence emission intensity (quenching) of the donor.

FRET-based reagents and methods are widely used in nucleic acid hybridization assays. One example of a homogeneous DNA hybridization assay format uses two oligonucleotide probes complementary to contiguous sequences of the target DNA. One probe carries a donor fluorophore on the 3′-end, the other an acceptor fluorophore on the 5′-end, so that when the two probes hybridize to the target DNA, the two fluorophores are adjacent to each other and FRET occurs. Hybridization is thus signaled by a decrease in the donor emission and a rise in the acceptor emission [Heller, M J & Morrison, L E, Chemiluminescent and fluorescent probes for DNA hybridization. In Rapid Detection and Identification of Infectious Agents. Edited by Kingsbury D T Falkow S, New York, Academic Press 1985: 245-256].

Another approach uses two complementary oligonucleotide strands, in which one strand is labeled on the 5′-end with fluorescein and the complementary strand is labeled on the 3′-end with a quencher of fluorescein emission. Such probes are able to detect unlabeled target DNA by competitive hybridization, producing fluorescence signals that increase with increasing DNA target concentration [Morrison et al. Solution-phase detection of polynucleotides using interacting fluorescence labels and competitive hybridization, Anal. Biochem. 1989, 183:231-244]. Another version of this type of “quench-release” assay employs probes called “molecular beacons”. These probes are single stranded oligonucleotides that possess a stem-loop structure. The loop portion of the probe is a sequence complementary to a predetermined sequence in a target nucleic acid. The stem is formed by the annealing of two complementary arm sequences that are on either side of the loop portion. A fluorophore is attached to one end of one arm and a non-fluorescent quencher is attached to the end of the other arm. The stem brings the fluorophore and the quencher close together. The hybrid formed by the probe with the target sequence is longer and more stable than the stem formed by the arm sequences. Thus, binding of the probe to the target extends the loop structure so that the fluorophore and the quencher are far from each other and fluorescence is no longer quenched [Tyagi S & Kramer F R, Molecular beacons-probes that fluoresce upon hybridization. Nat. Biotechnology 1996, 14:303-308].

Another example of a quench-release assay is provided by real-time PCR (polymerase chain reaction) 5′exonuclease assays. In this case a specific oligonucleotide probe is annealed to a target sequence located between the two primer sites. The probe is labeled with a reporter fluorophore at the 5′-end and a quencher fluorophore in the middle, or at the 3′-end. When the probe is intact, the reporter dye emission is quenched owing to the physical proximity of the reporter and quencher. Cleavage of the probe by 5′-3′ exonuclease activity of Taq polymerase during strand elongation releases the reporter from the oligo probe and thus its proximity to the 3′ quencher, resulting in an increase in reporter emission intensity. Thus, after each PCR cycle the observed fluorescence increases. The cycle at which the emission intensity of the sample rises above baseline is inversely proportional to the initial target sequence concentration [Holland, et al. Detection of specific polymerase chain reaction product by utilizing the 5′-3′ exonuclease activity of Thermus aquaticus DNA polymerase. Proc. Nat. Acad. Sci. USA 1996, 93:5395-5400.

The use of FRET-based or quench-release methods in assay design is not limited to detection of nucleic acids. FRET systems can be designed, for example, to detect binding of a ligand to a protein. FRET has also been exploited in the assay of enzymes or similar catalytic species based on the ability of the analyte to cleave a chemical bond linking a FRET donor-acceptor pair. For example, a protease can be assayed by monitoring the decrease in energy transfer efficiency (increase in donor fluorescence emission) between donor and acceptor linked together by a peptide fragment. As the linkage is broken the donor and acceptor become separated and efficient transfer of energy is no longer possible. This technique has been used to design gene reporter assays.

Another class of FRET assays is based on the use of tandem fusions of green fluorescent proteins (GFP) to form a donor-acceptor pair. An example is a calcium indicator whose structure is based on a cyan-emitting GFP (CFP) separated from a yellow-emitting GFP (YFP) by the calmodulin Ca²⁺-binding protein (CaM) and a calmodulin-binding peptide. If Ca²⁺ ions are bound, CaM wraps around M13, and the construct forms a more compact shape, leading to a higher efficiency of excitation transfer from the donor CFP to the acceptor YFP. [Miyawaki et al. Dynamic and quantitative Ca²⁺ and Ca²⁺-calmodulin in intact cells. Proc. Nat. Acad. Sci. USA 1999, 96:2135-2140].

However, FRET assays in their current form, which determine FRET efficiency from the ratio of sensitized acceptor fluorescence to donor fluorescence suffer from one important drawback. The problem is that, the absorption spectra of GFPs have long tails on the short-wavelength (blue) side and their emission spectra have long tails on the long-wavelength (red) side. This results in a cross-talk problem.

The difficulty arises because the donor and acceptor excitation bands overlap, making it impossible to excite only the donor. Moreover, the donor and acceptor emission bands also overlap, making it impossible to detect only the acceptor fluorescence. Instead, when the donor is excited, there is also some direct excitation of acceptor, and the detected signal contains not only sensitized acceptor fluorescence, but also directly excited acceptor fluorescence as well as a contribution from donor fluorescence. Thus, the FRET detection channel (defined by the detection spectral bandpass) has contributions from three signals, only one of which is related to FRET. The cross-talk contributions to the FRET channel can be a significant fraction of the detected signal. This can limit the sensitivity of these assays and requires cumbersome and unreliable corrections, which might not be feasible in the ISS environment.

It is clear from this brief review that a large variety of luminescent assays are known in the art, which rely on the use of FRET or quenching and use steady state intensity-based fluorescence detection.

Despite the advantage of being homogeneous, many FRET and quenching assays based on steady-state intensity detection are relatively insensitive and suffer from limited dynamic range. These limitations may arise from spectral crosstalk, as described above for FRET assays, or in the case of quenching assays because steady-state intensity measurements may not be able to differentiate between the quenched and unquenched species. For instance, when the quenched species is initially present in large excess, the quenched species may luminesce with a smaller quantum yield than the unquenched form but still contribute significant luminescence to the detected signal. This luminescence from the unquenched species can limit sensitivity and dynamic range.

An example of this is real-time PCR as described above. This assay starts with a large excess of the quenched form of the luminophore and as the amplification process progresses (separation of luminophore from quencher) the amount of the unquenched form increases. This results in a progressive increase in luminescent signal at each PCR cycle. In this assay, the quantity of interest is the total amount of unquenched species after each cycle. However, with conventional steady-state intensity detection what is measured at any time is the scalar sum of the intensities of the quenched and unquenched species and a positive result is not obtained until the total signal from both quenched and unquenched forms of the luminophore exceeds (by a statistically significant amount based on the noise characteristics of the detection system) the baseline luminescence generated by the low-quantum-yield quenched species initially present in large excess. Therefore, the luminescence generated by the lower quantum yield but higher concentration quenched species limits the detection sensitivity. These limitations of current FRET or quench-release assays may be circumvented with the use of frequency-domain fluorometry detection instead of steady-state fluorescence detection.

Steady-state detection methods measure the intensity of the luminescence signal in a selected spectral band. In the FRET and quench release assays described, the emission of the quenched and unquenched species are spectrally indistinguishable. Thus their separate contributions to the total signal amplitude cannot be discerned by steady-state detection methods. However, the quenched and unquenched species often differ in fluorescence lifetime. Thus, a detection method that is sensitive to changes in fluorescence lifetime can provide a means to discriminate between the quenched and unquenched species. (Principles of Fluorescence Spectroscopy, J. R. Lakowicz, Second Edition, Plenum Publishers, 1999, p.623). For example, in the case of real-time PCR described above, lifetime discrimination could be used to assess the separate contributions of the quenched and unquenched species to the total fluorescence signal with a resultant improvement in sensitivity.

It is an object of the present invention to phase fluorometry detection to exploit the difference in fluorescence lifetime of the quenched and unquenched species in FRET and quenching assays to obviate or mitigate the above mentioned limitations and disadvantages of steady-state intensity detection, and hence to improve the sensitivity of these types of assays.

### SUMMARY OF THE INVENTION

The present invention is an apparatus and method, using phase fluorometry, to improve the sensitivity of fluorescence assays in which the detected fluorescence signal contains, in addition to the analytical fluorescence signal of interest, contributions from another fluorescing species in the sample that is not spectrally separable from the analytical signal of interest. In such assays, the fluorescence from this other species constitutes a background interference that limits sensitivity. When the concentration of the interfering species changes during the assay, it is not possible to subtract the background with conventional steady-state intensity measurements. The present invention employs phase sensitive detection to provide a means to separately assess the contributions from the analytical and the background signals, and hence to remove the interfering background signal, when the fluorescence lifetimes of both the analytical species and the interfering species are known

### BRIEF DESCRIPTION OF THE DRAWINGS

The present invention may be better understood and its numerous objects and advantages will become apparent to those skilled in the art by reference to the accompanying drawings in which:

FIG. 1 is a schematic diagram of a phase fluorometer operating in accordance with the method of the present invention; and

FIG. 2 is a graph illustrating the relationship between the phase and amplitude of the emitted luminescence measured by the fluorometer of FIG. 1, and the phase and amplitude of the two luminescent species.

### DETAILED DESCRIPTION OF THE PREFERRED EMBODIMENT

The invention is preferably practiced with a FRET-based, quenching or quench-release assay in which there are two luminescent species with different lifetimes whose spectral signals overlap within the single pass band of the detector. Detection can be implemented with any luminescence phase-sensitive detection system with the appropriate resolution. One preferred embodiment would combine a FRET-based or quench-release molecular recognition assay with the phase-sensitive detection system described in U.S. Pat. No. 5,818,582, incorporated hereby by reference.

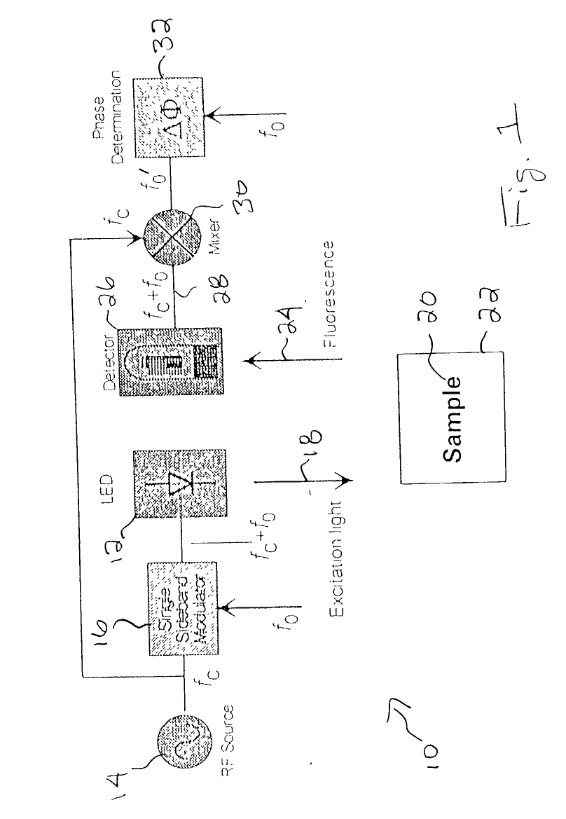

With reference to FIG. 1, a phase detection system 10 employing the subject method includes a light source 12, such as a laser diode or a light-emitting diode. Alternatively, the detection system may include a CW laser with an external modulator, such as an argon ion laser modulated with a Pockels cell, or any other light source whose amplitude can be modulated in the RF frequency range. A low frequency baseband signal fₒ, produced by a baseband frequency generator (not shown), is up-converted by combination with a high frequency carrier signal f_{c}, produced by a carrier frequency generator 14, in a single sideband modulator 16. The composite signal (f_{c}+fₒ) is used to directly modulate the light source 12, with the excitation light 18 emitted by the light source 12 being used to excite a sample 20 residing in the sample container 22. The fluorescence 24 emitted by the sample 20 acquires a phase delay corresponding to a frequency-weighted average of the lifetimes of the species in the sample 20.

The emitted fluorescence 24 is detected by a detector 26, for example a photomultiplier tube (PMT). The signal 28 from the detector (f_{c}+fₒ) is down-converted in a mixer 30 by subtracting the carrier signal f_{c}. The resultant signal fₒ′, which retains the phase information resulting from the interaction between the fluorescence and the sample, is compared to the baseband signal fₒ and the phase and/or amplitude difference is determined 32.

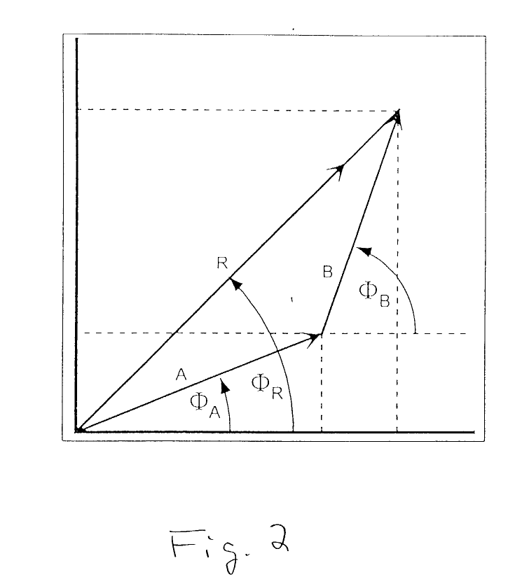

In accordance with the present invention, a sample 20 in which a molecular recognition assay is to be performed is illuminated with excitation light 18 modulated at a high frequency appropriate to the luminescence lifetimes of interest (ω≈1/τ, where ω=2πf, π is the luminescence lifetime, and f is the modulation frequency). Under modulated excitation the emitted luminescence 24 is also amplitude modulated at the same frequency but is delayed in phase relative to the excitation light 18 due to the finite duration of the absorption-emission process. When the sample 20 contains two separate luminescent components with different but known luminescent lifetimes τ_{A} and τ_{B} (e.g., a quenched and unquenched species in a FRET or quench-release assay) corresponding to phase angles Φ_{A} and Φ_{B}, the system 10 will measure an amplitude, R, and a phase Φ_{R} which represent the vector sum of the individual components as illustrated in FIG. 2. From knowledge of the measured amplitude R and phase Φ_{R} and the known phase angles Φ_{A} and Φ_{B} that correspond to the known fluorescence lifetimes of the quenched and unquenched species respectively, the amplitudes A or B of the unquenched and quenched signals can be calculated from the following trigonometric expressions.

R cos Φ_{R}=A cos Φ_{A}+B cos Φ_{B}

R sin Φ_{R}=A sin Φ_{A}+B sin Φ_{B}

It should be appreciated that the interaction between the first binding agent, labeled with a donor luminophore in a first case or a quenched luminophore in a second case (the luminophore having a known luminescence lifetime τ), and the second binding agent produces a mixture of bound first and second binding partners, unbound first binding partners, and unbound second binding partners. It should also be appreciated that the mixture has an initial ratio of bound binding partners to unbound binding partners which may be measured and that the luminescence lifetime of the donor luminophore in the first case and the quenched luminophore in the second case is changed to τ′ by the binding of the first binding partner to the second binding partner. The assays cause a change in the ratio of bound binding partners to unbound binding partners, thereby changing the ratio of τ to τ′.

Illuminating the sample with a sinusoidally modulated light having a frequency, f≈½πτ, produces a detectable phase shift in the emitted luminescence. The luminescence emission detected by the system 10 contains contributions primarily from donor luminophores of bound binding partners and unbound binding partners. Measuring the amplitude and phase of the luminescence signal allows the amplitude and phase of the luminescence signals of donor luminophores of bound binding partners and unbound binding partners to be calculated using vector addition, as illustrated in FIG. 2.

### EXAMPLES

1. FRET-based or quench-release molecular recognition assay in which 1) one of the molecular partners is labeled with a donor luminophore and the other is labeled with an acceptor luminophore or a non-luminescent quencher, 2) the molecular recognition event of interest causes a discrete change in FRET or quenching efficiency, 3) the luminescence lifetimes of the high- and low-efficiency FRET or quench states are known 4) phase detection, as described above, and signal processing according to this invention to remove luminescence background from the quenched or high-efficiency FRET species.

2. A FRET-based or quench-release assay in which 1) one of the molecular partners is labeled with a luminophore, 2) the molecular recognition event of interest causes a discrete change in the luminescence lifetime of the luminophore, 3) the luminescence lifetimes of both the unperturbed and the perturbed states of the luminophore are known, 4) phase detection, as described above, and signal processing according to this invention to remove luminescence background due to emission from the unperturbed species.

3. An assay, as described in 1 or 2 above, in a homogeneous solution format in which both molecular recognition partners are mixed in solution in a container, such as a well in a microwell plate. Molecular recognition partners include but are not limited to small organic molecules, peptides, proteins, antibodies, enzymes, nucleic acids, peptide nucleic acids (PNAs), aptamers, lipids and carbohydrates.

4. An assay, as described in 1 or 2 above, in a heterogeneous format in which one of the molecular recognition partners is immobilized on a solid-phase matrix and the other partner is in a solution that comes into contact with the solid phase. Such solid phase matrices include, but are not limited to, plastic beads, polymeric membranes, the bottom or walls of wells in a microwell plate, glass surfaces, surfaces of waveguides in evanescent-wave excitation assays and to microarray chips, such as DNA arrays, RNA arrays, protein arrays, peptide arrays, antibody arrays, aptamer arrays and PNA arrays.

5. An assay as described in 3 above in which the solid phase is coated with a thin film of metal suitable to perform surface plasmon resonance (SPR) measurements of molecular interaction between the recognition partners simultaneously or in tandem with luminescence detection. The gold coated surface may be smooth and configured in a Kretschmann or Otto configuration for SPR measurements or can be a metal-coated grating for use in grating-coupled SPR measurements

While preferred embodiments have been shown and described, various modifications and substitutions may be made thereto without departing from the spirit and scope of the invention. Accordingly, it is to be understood that the present invention has been described by way of illustration and not limitation.

What is claimed is:

1. A method for improving the sensitivity of fluorescence resonance energy transfer assays comprising the steps of:
adding a quantity of a first binding partner to a sample container, the first binding partner being labeled with a donor luminophore having a known luminescence lifetime τ;
adding a quantity of a second binding partner to the sample container, a portion of the second binding partner binding with a portion of the first binding partner to produce a mixture of bound first and second binding partners, unbound first binding partners, and unbound second binding partners, the mixture having an initial ratio of bound binding partners to unbound binding partners, the luminescence lifetime of the donor luminophore labeling the first binding partner being changed to τ′ by the binding of the first and second binding partners;
adding the reagents to the sample container to produce a sample, the addition of the reagents causing a change in the ratio of bound binding partners to unbound binding partners;
illuminating the sample with a sinusoidally modulated light having a frequency, f, where f≈½πτ, the light producing a detectable phase shift in the emitted luminescence;
detecting the luminescence emission of the donor luminophore with a single-frequency phase fluorometer system, the luminescence emission containing contributions from donor luminophores of bound binding partners and unbound binding partners;
measuring the amplitude and phase of the luminescence signal; and
calculating the amplitude and phase of the luminescence signals of donor luminophores of bound binding partners and unbound binding partners from the measured amplitude and phase of the detected luminescence signal and from the known or separately measured phase of the luminescence signals of donor luminophores of bound binding partners and unbound binding partners using vector addition.

2. A method for improving the sensitivity of quench-release fluorescence assays comprising the steps of:
adding a quantity of a first binding partner to a sample container, the first binding partner being labeled with a quenched luminophore having a known luminescence lifetime τ;
adding a quantity of a second binding partner to the sample container, a portion of the second binding partner binding with a portion of the first binding partner to produce a mixture of bound first and second binding partners, unbound first binding partners, and unbound second binding partners, the mixture having an initial ratio of bound binding partners to unbound binding partners, the luminophore being unquenched by the binding of the first and second binding partners whereby the luminescence lifetime of the luminophore labeling the first binding partner is changed to known luminescence lifetime τ′;
adding the reagents to the sample container to produce a sample, the addition of the reagents causing a change in the ratio of bound binding partners to unbound binding partners;
illuminating the sample with a sinusoidally modulated light having a frequency, f, where f≈½πτ, the light producing a detectable phase shift in the emitted luminescence;
detecting the luminescence emission of the quenched and unquenched luminophores with a single-frequency phase fluorometer system;
measuring the amplitude and phase of the luminescence emission; and
calculating the amplitude and phase of the luminescence emissions of quenched and unquenched luminophores from the measured amplitude and phase of the detected luminescence emission and from the known or separately measured phase of the emissions of the quenched and unquenched luminophores using vector addition.

3. A method for improving the sensitivity of real-time polymerase chain reaction 5′exonuclease assays comprising the steps of:
adding a quantity of a first binding partner to a sample container, the first binding partner being labeled with a quenched luminophore having a known luminescence lifetime τ;
adding a quantity of a second binding partner to the sample container, a portion of the second binding partner binding with a portion of the first binding partner to produce a mixture of bound first and second binding partners, unbound first binding partners, and unbound second binding partners, the mixture having an initial ratio of bound binding partners to unbound binding partners, the luminophore being unquenched as a consequence of the binding of the first and second binding partners whereby the luminescence lifetime of the luminophore labeling the first binding partner is changed to known luminescence lifetime τ′;
adding the reagents to the sample container to produce a sample, the addition of the reagents causing a change in the ratio of quenched luminophores to unquenched luminophores;
illuminating the sample with a sinusoidally modulated light having a frequency, f, where f≈½πτ, the light producing a detectable phase shift in the emitted luminescence;
detecting the luminescence emission of the quenched and unquenched luminophores with a single-frequency phase fluorometer system;
measuring the amplitude and phase of the luminescence emission; and
calculating the amplitude and phase of the luminescence emissions of quenched and unquenched luminophores from the measured amplitude and phase of the detected luminescence emission and from the known or separately measured phase of the emissions of the quenched and unquenched luminophores using vector addition.

4. A method for conducting functional cell assays based on fluorescence resonance energy transfer in a sample of cells that have specific components labeled with genetically encoded donor and acceptor fluorescent protein tags, a portion of the donor fluorescent protein tags being associated with an acceptor fluorescent protein tags and a portion of the donor fluorescent protein tags remaining free, the fluorescence lifetime τ of the free donor fluorescent protein tag and the fluorescence lifetime τ′ of the acceptor-associated donor fluorescent protein tag being known, the method comprising the steps of:
(a) illuminating the sample cells with light in a wavelength range to preferentially excite the donor fluorescent protein tags, the light being sinusoidally modulated at a frequency, f, where f≈½πτ, the light producing a detectable phase shift in the emitted luminescence;
(b) detecting the luminescence emissions of the free donor fluorescent protein tags and the acceptor-associated donor fluorescent protein tags with a single-frequency phase fluorometer system;
(c) measuring the amplitude and phase of the luminescence emission;
(d) calculating the amplitude and phase of the luminescence emissions of the free donor fluorescent protein tags and the acceptor-associated donor fluorescent protein tags from the measured amplitude and phase of the detected luminescence emission and from the known or separately measured phase of the emissions of the free donor fluorescent protein tags and the acceptor-associated donor fluorescent protein tags using vector addition;
(e) adding a test compound to the sample of cells to elicit a cellular response based on the change in the number of free donor fluorescent protein tags and the number of acceptor-associated donor fluorescent protein tags;
(f) repeating steps (b) through (d); and
(g) assigning a value to the cellular response based on the change in the amplitudes of the fluorescence of the free donor fluorescent protein tags and the acceptor-associated donor fluorescent protein tags that occurs in response to addition of the test compound.

5. The method of claim 4 wherein step (a) includes suspending the sample cells in a volume of fluid to form a suspension of sample cells.
